(19) United States
(12) Patent Application Publication
(10) Pub. No.: US 2022/0111112 A1
(43) Pub. Date: Apr. 14, 2022

(54) Flexible Gelatin Sealant Dressing with Reactive Components

(71) Applicant: Ethicon, Inc., Somerville, NJ (US)

(72) Inventors: Ashley DeAnglis, Skillman, NJ (US); Doug Johns, Milford, NJ (US); Guanghui Zhang, Belle Mead, NJ (US); Wilai Kosol, Somerville, NJ (US)

(21) Appl. No.: 17/559,447

(22) Filed: Dec. 22, 2021

Related U.S. Application Data
(62) Division of application No. 16/738,294, filed on Jan. 9, 2020.

(51) Int. Cl. A61L 15/32; A61L 15/22; A61L 15/42

(52) U.S. Cl. CPC A61L 15/325; A61L 24/0094; A61L 15/425; A61L 15/225

(57) ABSTRACT

The present invention is directed to hemostatic sealants having a compressed porous substrate, an electrophilic group containing component that is not gelatin or collagen, a nucleophilic group containing component, and a buffering agent. The present invention also relates to method for manufacture and use of such sealants to seal and/or achieve hemostasis.

### BACKGROUND

Absorbable hemostatic patches containing two cross-linkable components have been described in the literature including in US Publication No. 2011/0045047 A1. The cross-linkable components for such patches can be a pair of coreactive compounds or a substrate coated with a coreactive compound having available units that can form covalent crosslinks with the corresponding coreactive group on the substrate. Gelatin and collagen substrates have been combined with reactive components as hemostatic and sealing wound dressings.

Applicants discovered that selected gelatin matrices that were coated with a combination of polyethylene glycol (PEG)-based crosslinkers can achieve strong adhesion to tissue thus enabling rapid hemostasis and sealing. Compressed gelatin matrix was found to be flexible and conformable to the tissue, relative to conventional gelatin sponge (approximately 1 cm thick), and can achieve hemostasis in several severe bleeding models

### SUMMARY OF THE INVENTION

The present invention is directed to hemostatic sealants based on a compressed porous substrate, an electrophilic group containing component that is not gelatin or collagen, a nucleophilic group containing component and a buffering agent. The compressed porous substrate can be a layer of collagen or gelatin, wherein the gelatin can be crosslinked. Preferably, the gelatin has a thickness of less than 5 millimeters, more preferably a thickness of 2 millimeters or less. In one embodiment, the gelatin material has an open cell pore structure throughout.

The electrophilic component can be a polymeric compound derived from a polyethylene glycol having at least two electrophilic groups, wherein the electrophilic groups can be selected from the group consisting of succinimides, succinimidyl glutarate (SG), carboxymethyl-hydroxybutyrate-N-hydroxysuccinimide, carbonyldiimidazole, sulfonyl chloride, aryl halides, sulfosuccinimide ester, epoxide, aldehyde, maleimides and imidoester and combinations thereof.

The nucleophilic group containing component can be a polymeric material derived from polyethylene glycol having at least two nucleophilic groups, wherein the nucleophilic groups can be selected from the group consisting of hydroxyl, thiol, amine and combinations thereof. In one embodiment, the electrophilic, nucleophilic and buffering components can each be a milled powder.

The present invention is also directed to methods for sealing tissue by adhering the hemostatic sealants as described above to a moist tissue surface.

The present invention is also directed to methods for achieving hemostasis by adhering the hemostatic sealants described above to a tissue exhibiting severe or oozing bleeding.

The present invention is also directed to methods for manufacturing a hemostatic sealants by suspending an electrophilic group containing component that is not gelatin or collagen, a nucleophilic group component and a buffering agent in an inert, non-aqueous solvent and coating a compressed porous substrate with the suspension. In an alternative embodiment, such methods can further include compressing a crosslinked porous gelatin substrate into a compressed porous substrate.

In a preferred embodiment, the present invention is directed to a compressed gelatin matrix, preferably to a thickness of about 0.2 cm prior to application, that has been coated with an electrophilic reactive containing polyethylene glycol, preferably PEG-N-hydroxysuccinimide (PEG-NHS) and a nucleophilic reactive containing polyethylene glycol, preferably PEG-Amine, each preferably in the powder, particle or aggregate form that are substantially non-reactive in the absence of moisture, along with a buffering agent.

The compressed gelatin matrix has optimal physical properties for a hemostatic matrix. The compressed and coated gelatin matrix is flexible while retaining greater strength compared to that of a non-compressed and uncoated gelatin matrix. The compressed matrix has higher cross-linking density compared to a conventional matrix thus allowing higher flexibility without breakage. Unlike gelatin films, the compressed gelatin matrix has a porous structure that allows the reactive components to penetrate beyond the outer surface of the matrix and upon reaction to further integrate within the structure of the matrix and with the adjacent tissue surface. The benefit of this integration is that the powder can interact with more surface area of the matrix thus providing greater chemical bonding between the matrix and sealant components.

Additionally, this integration allows the matrix to augment the sealant strength by proving additional mechanical support. Finally, the compressed matrix has more amine groups available at the surface interface which permits more covalent crosslinking for better adhesion and better mechanical strength than a conventional gelatin matrix.

### DETAILED DESCRIPTION

The dressing of the present invention includes a compressed biomaterial carrier layer that contains coreactive, crosslinkable components. In a still further alternative embodiments, the two coreactive components can be applied in liquid, powder, or combinations thereof, onto the biomaterial substrate. In each instance for the production of packaged and ready-to-use embodiments, the co-reactive, crosslinkable components must be applied in a manner to prevent reaction prior to application onto tissue. In an alternative embodiment, one or more of the coreactive, crosslinkable components can be applied onto the matrix in the surgical setting shortly before application of the dressing onto a tissue surface, an in-situ formed embodiment.

In one embodiment, the substrate is made from layers of biomaterials selected from the group consisting of a biomaterial, preferably a protein, a biopolymer or a polysaccharide matrix, especially a collagen, gelatin, fibrin, starch or chitosan matrix. Preferably, the matrix of the present invention is biodegradable, i.e. it is naturally absorbed by the patient's body after some time. In any way, the material (including the matrix) must be biocompatible, i.e. have no harming effect to the patient to whom the material is administered. Such biodegradable materials are specifically suitable in situations where hemostasis is achieved inside the body, i.e. in the course of surgery and the site is closed after surgery.

Accordingly, in one embodiment, the substrate is preferably a biomaterial selected from biopolymers such as a protein, or a polysaccharide. Especially preferred is a biomaterial selected from the group consisting of collagen, gelatin, fibrin, a polysaccharide, e.g. hyaluronic acids, chitosan, and a derivative thereof, more preferred gelatin, collagen and chitosan, especially preferred gelatin and collagen. Such gelatin or collagen matrix used for the present invention can be derived from any collagen suitable to form a gel, including a material from liquid, pasty, fibrous or powdery collagenous materials that can be processed to a porous or fibrous matrix as well as particles. The preparation of a collagen gel for the production of a sponge or sheet may include acidification until gel formation occurs and subsequent pH neutralization. To improve gel forming capabilities or solubility the collagen may be (partially) hydrolyzed or modified, as long as the property to form a stable sponge or sheet when dried is not diminished.

Collagen and gelatin-containing embodiments in accordance with the present disclosure include a porous substrate having a first coreactive and crosslinkable component applied to a first portion of the porous substrate and a second coreactive and crosslinkable component applied to a second portion of the porous substrate.

The porous substrate of the dressing has openings or pores over at least a portion of a surface thereof. As described in more detail below, suitable materials for forming the porous substrate include, but are not limited to fibrous structures (e.g., knitted structures, woven structures, non-woven structures, etc.) and/or foams (e.g., open or closed cell foams). In embodiments, the pores may be in sufficient number and size so as to interconnect across the entire thickness of the porous substrate.

Woven fabrics, knitted fabrics and open cell foam are illustrative examples of structures in which the pores can be in sufficient number and size so as to interconnect across the entire thickness of the porous substrate. In embodiments, the pores do not interconnect across the entire thickness of the porous substrate. Closed cell foam or fused non-woven materials are illustrative examples of structures in which the pores may not interconnect across the entire thickness of the porous substrate. The pores of the foam porous substrate may span across the entire thickness of porous substrate. In yet other embodiments, the pores do not extend across the entire thickness of the porous substrate, but rather are present at a portion of the thickness thereof. In embodiments, the openings or pores are located on a portion of the surface of the porous substrate, with other portions of the porous substrate having a non-porous texture.

Where the porous substrate is fibrous, the porous substrate may be formed using any method suitable to forming fibrous structures, including but not limited to knitting, weaving, non-woven techniques, wet-spinning, electro-spinning, extrusion, co-extrusion, and the like. Suitable techniques for making fibrous structures are within the purview of those skilled in the art. In embodiments, the textile has a three dimensional structure, such as the textiles described in U.S. Pat. Nos. 7,021,086 and 6,443,964, the disclosures of which are incorporated herein by this reference in their entirety.

Where the porous substrate is a foam, the porous substrate may be formed using any method suitable to forming a foam or sponge including, but not limited to the lyophilization or freeze-drying of a composition or introducing gaseous components or gas-generating components into the composition. The foam may be cross-linked or non-cross-linked, and may include covalent or ionic bonds, or physical entanglements. Suitable techniques for making foams are within the purview of those skilled in the art.

The biomatrix substrate is compressed to a thickness of less than 5 millimeters, preferably 2 millimeters or less. The compression step can be done by putting a foam between two parallel plates and applying a compression force for a time period effective to achieve the desired thickness. The compression step can also be accomplished through roller-compression by inserting a form between two rotating cylindrical bodies with a defined gap effective to obtain the desired thickness. If cross-linking of the foam is needed, the compression step can be done either before or after the cross-linking step. Preferably, the compression step is done before the cross-linking step.

Reactive PEG components are applied onto each matrix in the same manner. The two reactive PEG components and pH modifier (buffering agent), while in powder form, are suspended together in twenty (20) mL of an organic solvent, available from 3M™ as Novec™ 7000 Engineered Fluid, which is 1-methoxyheptafluoropropane (HFE-7000), vortexed to mix. The resulting suspension is dispensed onto a major (in terms of exposed surface area) surface of the matrix using a pipette. The resulting coated matrices are allowed to dry for at least one (1) hour in a ventilated chemical fume hood.

The following non-limiting examples are provided to illustrate certain aspects of the desired compressed and coated matrix.

### EXAMPLE 1

Three gelatin matrix types were tested, listed below. These three matrices are absorbable commercial grade hemostatic sponges or films that are sterile, water-insoluble, porcine-derived matrices intended for hemostatic use by applying to a bleeding surface.
1) Gelatin sponge (10 mm thickness), SURGIFOAM® Absorbable Gelatin Sponge, size 100, 8 cm×12.5 cm, Product code 1974, commercially available from Ethicon, Inc.
2) Compressed gelatin sponge (2 mm thickness) SURGIFOAM® Absorbable Gelatin Sponge, size 100 c, 8 cm×12.5 cm, Product code 1975, commercially available from Ethicon, Inc.
3) Gelatin film (0.5 mm thickness), SPONGOSTAN® Absorbable Hemostatic Gelatin, size 20 cm×7 cm, commercially available from Ethicon, Inc.

The total powder coverage area is applied to provide 20 mg powder/cm² on the major surface of matrix. Milling for each component is performed individually using a porcelain mortar and pestle to achieve a fine powder. The milling process is performed in a sealed chamber filled with nitrogen gas such that the moisture level is less than 25%. The individual power composition per unit area is, on average, on a dry powder basis:
15 mg/cm² PEG-SG4 (4-arm PEG-SG, molecular weight=10,000 Da)
4 mg/cm² of PEG-Amine (4-arm, molecular weight=4,000 Da)
1 mg/cm² sodium bicarbonate (pH modifier/buffering agent)
   To coat a matrix with an exposed surface area of one hundred (100) cm², the following amounts of powder were put into suspension:
1500 mg milled PEG-SG4
400 mg milled 4-arm PEG-Amine
100 mg sodium bicarbonate powders

Milling for each component is performed individually using a porcelain mortar and pestle to achieve a fine powder. The milling process is performed in a sealed chamber filled with nitrogen gas such that the moisture level is less than 25%. The powder particles are on average approximately 100 microns or less for all powders, as measured by light microscopy.

Results: The uncompressed gelatin sponge coated as described above was stiff, inflexible and cracked when force was applied. The combination of thickness and rigidity/inflexibility limits the ability of this matrix to conform to tissue and make direct contact without fracturing. Conversely, the gelatin film has a smooth surface and does not absorb the suspended particles, thus resulting in very high friability and less powder retained on the surface of the matrix. The compressed gelatin sponge, in contrast, is more flexible and has greater strength compared with that of standard Surgifoam. The compressed sponge retains its powder with little to no friability.

### EXAMPLE 2

### Adherence of the Compressed Gelatin Matrix to Tissue

The compressed gelatin matrix is prepared as described in Example 1. The coated matrix is evaluated for its ability to adhere to freshly harvested porcine spleen tissue using the following assessment method.

A freshly harvested porcine spleen is utilized for testing. Prior to testing, the spleen is maintained at room temperature and rinsed with saline. The surface of the spleen is kept moist with saline during the entire testing session. The procedure for testing each sample is as follows: 1) A sample of dimension 1 inch×1 inch is precut prior to testing; 2) 1 mL of saline (dispensed from a 3 mL syringe) is sprayed through an atomizer onto the area of sample application; 3) the sample is applied to the wetted area and wet gauze pads are applied to the back of the sample for 2 minutes holding firm, even manual pressure; 4) After 2 minutes, the wet gauze is removed and the edge of the sample is peeled from the spleen surface using forceps; 5) The force required to remove the sample is assessed on a semi-quantitative scale with 0 being no adhesion (lifts with no resistance) to 3 being very strong adhesion (difficult to peel away and lifts spleen while removing).

The PEG-coated compressed gelatin sponge could not be removed without damaging the gelatin matrix. A thin layer of sealant integrated into the matrix remained adhered to the tissue when the matrix was forcibly peeled away. An adhesion score of 3 was assigned (scale of 0-3).

To assess adhesion in a more severe model, the PEG-coated compressed gelatin matrix is tested for adhesion in an ex vivo spleen resection model. To demonstrate the ability of the powder to absorb into the matrix material, the PEG-coated compressed gelatin matrix is shaken to remove any loose/friable powder. A complete resection is made 3 inches from the end of the spleen. The PEG-coated matrix (with loose powder removed) is wrapped around the cut end of the spleen, and contacted the top, bottom and cut surfaces. After 2 min of compression, the matrix adheres well to the tissue and conforms to the tissue surrounding the cut surface. An adhesion score of 3 is assigned (scale of 0-3). In summary, the compressed gelatin matrix has optimal properties in terms of thickness, flexibility, strength and the ability to retain powder compared to other gelatin matrices evaluated.

### EXAMPLE 3

### PEG-coated onto Compressed Gelatin Matrix Tested in Three Severe Porcine Bleeding Models

The performance of PEG-coated onto compressed gelatin sponges is evaluated in three severe bleeding models.

### Animal Models

Testing is performed by a trained veterinary surgeon. Three porcine bleeding models are employed to assess the performance of PEG-coated compressed gelatin sponge prototypes. To produce a severe model, a bolus dose of unfractionated heparin is administered to the animal prior to testing and additional doses are given throughout the procedure to maintain the Activated Clotting Time (ACT) level at 1.5-3.0 times above baseline. The sequence of testing is: 1) spleen biopsy punch model (8 mm diameter×5 mm deep); 2) spleen resection model; and 3) liver resection model. After creating each defect and applying the sample, tamponade is applied for 2 minutes with gauze. After relieving pressure, the site is monitored for bleeding for thirty (30) seconds. If bleeding is observed, an additional thirty to sixty (30-60) seconds of pressure is applied, and the site was re-evaluated.

### Test Article

PEG-SG4, PEG-Amine and bicarbonate powder are coated using HFE onto a compressed gelatin matrix as described in Example 1.

Spleen Biopsy Punch Model. The PEG-coated compressed gelatin matrix is applied to the spleen over a standardized punch defect (8 mm diameter and 5 mm deep). After two (2) minutes of tamponade with wet gauze, hemostasis is achieved. On adhesion testing, the test article adheres strongly to the tissue (score of 5 out of 5 as assessed by the surgeon). The test article cohesively separates from the edges on peeling, but a residual gelatin layer remains in contact with the tissue. The matrix remained adhered to the spleen throughout subsequent testing of the spleen at proximal sites.

Spleen Resection Model: The PEG-coated compressed gelatin sponge prototype is applied to the cut surface of the resected spleen (3.5 cm cut from earlier cut on end of spleen). Bleeding is characterized as very heavy with arterial spurting. When the test article described above is applied, a portion of the powder is dislodged from the matrix. After two (2) minutes of tamponade with wet gauze, hemostasis is achieved on the cut surface. The prototype is well adhered to the tissue. Liver Resection Model: The test article described above is applied to the cut surface of the resected liver (2.5 cm cut from earlier cut on end of liver). Bleeding is characterized as severe (the cut surface of liver had large cross-sectional area). After two (2) minutes of tamponade with wet gauze, hemostasis is achieved. No bleed through or edge oozing was observed. The test article is well adhered and the compressed gelatin matrix could be wrapped around the cut surface of the liver demonstrating its conformability.

1.-12. (canceled)

13. A method for manufacturing a hemostatic sealant comprising: a) suspending an electrophilic group containing component that is not gelatin or collagen, a nucleophilic group component and a buffering agent, all in powder format, in an inert, non-aqueous fluid; and b) coating a compressed porous substrate with the suspension, and optionally removing the fluid.

14. A hemostatic sealant according to claim 13 wherein the powder is milled.

15. A method according to claim 20 wherein the compressed porous substrate is a crosslinked porous gelatin.
